(19) United States
(12) Patent Application Publication
(10) Pub. No.: US 2023/0098586 A1
(43) Pub. Date: Mar. 30, 2023

(54) OPTOACOUSTIC PROBE FOR PROSTRATE IMAGING

(71) Applicant: Seno Medical Instruments, Inc., San Antonio, TX (US)

(72) Inventor: Jeff Harris, San Antonio, TX (US)

(73) Assignee: Seno Medical Instruments, Inc., San Antonio, TX (US)

(21) Appl. No.: 17/935,672

(22) Filed: Sep. 27, 2022

Related U.S. Application Data
(60) Provisional application No. 63/250,357, filed on Sep. 30, 2021.

(51) Int. Cl. A61B 8/00; A61B 5/00

(52) U.S. Cl. CPC A61B 8/42; A61B 5/0095

(57) ABSTRACT

A probe is provided for dual imaging of a tissue site that includes a light source configured to generate light that is transmitted along a light path to generate optoacoustic return signals and ultrasound return signals when the light reacts with the tissue site, and a transducer assembly including a first transducer on the distal end, and a second transducer on the distal end. The first transducer is configured to receive the optoacoustic return signals and having an acoustic lens provided over the first transducer, and the second transducer is configured to receive the ultrasound return signals.

### CROSS REFERENCE TO RELATED APPLICATION

This application claims priority to U.S. Provisional Application No. 63/250,357 (filed 30 Sep. 2021), The entire disclosure of that application is incorporated herein by reference.

### TECHNICAL FIELD

The present invention relates in general to the field of medical imaging, and in particular to a system relating to a probe for dual imaging.

### BACKGROUND

Optoacoustic (OA) imaging systems visualize thin tissue slices at a tissue site. A tissue site may contain a variety of tissue structures that may include, for example, tumors, blood vessels, tissue layers, and components of blood. In optoacoustic imaging systems, light is used to deliver optical energy to a planer slice of the tissue site, which as a result of optical absorption with the tissue structures, produce acoustic waves. An image spatially representing the tissue site can be generated by performing image reconstruction on acoustic signals that return to an ultrasound transducer array. Because biological tissue scatters impinging optical energy in many directions the optical energy can be absorbed by tissue structures outside of a targeted region, which can generate acoustic return signals that interferes with the imaging of tissue structures within the targeted region. Typically, the frequencies of signals obtained by OA imaging systems range between 250 MHz to 2.5 MHz.

Optoacoustic imagining systems can be utilized for targeted regions for a breast, prostrate, etc. In some applications, including for prostrate imaging, optoacoustic images can be supplemented with other imaging systems, including ultrasound imaging. Ultrasound imaging utilizes higher frequency transducers, up to 25 MHz, to obtain higher resolutions to help diagnosis and imaging guidance.

### BRIEF SUMMARY

New and useful systems, apparatuses, and methods for providing optoacoustic imaging are set forth in the appended claims. Illustrative embodiments are also provided to enable a person skilled in the art to make use the claimed subject matter.

Objectives, advantages, and a preferred mode of making and using the claimed subject matter may be understood best by reference to the accompanying drawings in conjunction with the following detailed description of illustrative embodiments.

In accordance with embodiments herein, a probe is provided for dual imaging of a tissue site. The probe has a distal end operable to contact the tissue site and a proximal end. The probe includes a light source configured to generate light that is transmitted along a light path to generate optoacoustic return signals and ultrasound return signals when the light reacts with the tissue site, and a transducer assembly including a first transducer on the distal end, and a second transducer on the distal end. The first transducer is configured to receive the optoacoustic return signals and having an acoustic lens provided over the first transducer, and the second transducer is configured to receive the ultrasound return signals. The probe also includes an optical window configured to carry light along the light path to the tissue site, and a microcontroller including one or more processors, and a memory coupled to the one or more processors. The memory stores program instructions, wherein the program instructions are executable by the one or more processors to convert the optoacoustic return signals from the first transducer into a first image, and convert the ultrasound return signals from the second transducer into a second image.

Optionally, the first transducer is spaced from the second transducer. In one aspect, the first transducer is 180° from the second transducer. In another aspect, the first transducer is stacked on the second transducer. In one example, the optoacoustic return signals received by the first transducer have a frequency range between 250 Hertz (Hz) and 2.5 Mega Hertz (MHz), and the ultrasound return signals have a frequency range between 20 MHz and 25 MHz. In another example, the light source is a laser. In yet another example, the first transducer extends further distally than the second transducer. In one embodiment, the probe also includes a triggering assembly coupled to the light source for actuating the light source.

In accordance with embodiments herein, a method of imaging a tissue site with a dual imaging probe is provided. The method includes placing a first transducer on a distal end of the dual imaging probe against a tissue site, actuating a light source for emitting light on the tissue site, and receiving, with the first transducer, optoacoustic return signals. The method also includes converting the optoacoustic return signals into an optoacoustic image, and rotating the dual imaging probe to place a second transducer on the distal end against the tissue site. The method also includes receiving, with the second transducer, ultrasound return signals, and converting the ultrasound return signals into an ultrasound image.

Optionally, rotating the dual imaging probe comprises rotating the dual imaging probe 180°. In one aspect, the optoacoustic return signals received by the first transducer have a frequency range between 250 Hertz (Hz) and 2.5 Mega Hertz (MHz), and the ultrasound return signals have a frequency range between 20 MHz and 25 MHz. In another aspect, rotating the dual imaging probe does not comprise withdrawing the dual imaging probe from the tissue site.

In accordance with embodiments herein, a probe for dual imaging is provided. The probe has a distal end operable to contact a tissue site and a proximal end, and the probe includes a light source configured to generate light that is transmitted along a light path to generate optoacoustic return signals and ultrasound return signals when the light reacts with the tissue site. The probe also includes a transducer assembly including a first transducer on the distal end, and a second transducer on the distal end. The first transducer is configured to receive the optoacoustic return signals in a first position, and the second transducer is configured to receive the ultrasound return signals in a second position. The probe also includes an optical window configured to carry light along the light path to the tissue site, and a microcontroller including one or more processors, and a memory coupled to the one or more processors. The memory stores program instructions, wherein the program instructions are executable by the one or more processors to convert the optoacoustic return signals from the first transducer into a first image, and convert the ultrasound return signals from the second transducer into a second image.

Optionally, the first transducer is spaced from the second transducer. In one aspect, the first transducer is 180° from the second transducer. In another aspect, the probe rotates 180° between the first position and the second position. In one example, the optoacoustic return signals received by the first transducer have a frequency range between 250 Hertz (Hz) and 2.5 Mega Hertz (MHz), and the ultrasound return signals have a frequency range between 20 MHz and 25 MHz. In another example, the light source is a laser. In yet another example, the first transducer extends further distally than the second transducer. In one embodiment, the probe also includes a triggering assembly coupled to the light source for actuating the light source.

### BRIEF DESCRIPTION OF THE DRAWINGS

The foregoing and other objects, features, and advantages of the invention will be apparent from the following more particular description of preferred embodiments as illustrated in the accompanying drawings, in which reference characters refer to the same parts throughout the various views. The drawings are not necessarily to scale, emphasis instead being placed upon illustrating principles of the invention.

FIG. 1 shows a schematic block diagram illustrating an embodiment of a combined optoacoustic and ultrasound system that may be used as a platform for the methods and devices disclosed herein.

FIG. 2A shows a side plan view of an embodiment of a probe that may be used in connection with the methods and other devices disclosed herein.

FIG. 2B shows a bottom plan view of an embodiment of a probe that may be used in connection with the methods and other devices disclosed herein.

FIG. 3 shows a side plan view of an embodiment of a probe that may be used in connection with the methods and other devices disclosed herein.

FIG. 4 shows a sectional view of a probe used in connection with the methods and other devices disclosed herein.

FIG. 5 is a schematic block diagram of a microcontroller utilized in connection with the methods and other devices disclosed herein.

FIG. 6 is a schematic block flow diagram of a method for imaging a tissue site with a probe for dual imaging in connection with the methods and other devices disclosed herein.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

The following description and drawings are illustrative and are not to be construed as limiting. Numerous specific details are described to provide a thorough understanding. However, in certain instances, well-known or conventional details are not described in order to avoid obscuring the description. References to one or an embodiment in the present disclosure are not necessarily references to the same embodiment; and such references mean at least one.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments, but not other embodiments.

The systems and methods are described below with reference to, among other things, block diagrams, operational illustrations and algorithms of methods and devices to provide optoacoustic imaging with out-of-plane artifact suppression. It is understood that each block of the block diagrams, operational illustrations and algorithms and combinations of blocks in the block diagrams, operational illustrations, and algorithms, can be implemented by means of analog or digital hardware and computer program instructions.

These computer program instructions can be stored on computer-readable media and provided to a processor of a general purpose computer, special purpose computer, ASIC, or other programmable data processing apparatus, such that the instructions, which execute via the processor of the computer or other programmable data processing apparatus, implements the functions/acts specified in the block diagrams, operational block or blocks and or algorithms.

In some cases, frequency domain-based algorithms require zero or symmetric padding for performance. This padding is not essential to describe the embodiment of the algorithm, so it is sometimes omitted from the description of the processing steps. In some cases, where padded is disclosed in the steps, the algorithm may still be carried out without the padding. In some cases, padding is essential, however, and cannot be removed without corrupting the data.

In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

Reference will now be made in more detail to various embodiments of the present invention, examples of which are illustrated in the accompanying figures. As will be apparent to one of skill in the art, the data structures and processing steps described herein may be implemented in a variety of other ways without departing from the spirit of the disclosure and scope of the invention herein and should not be construed as being limited to the embodiments set forth herein. Rather, these embodiments are provided so that this disclosure will be thorough and complete, and will fully convey the concept of the disclosure to those skilled in the art.

Embodiments herein may be implemented in connection with one or more of the systems and methods described in one or more of the following patents, publications, and/or published applications, all of which are expressly incorporated herein by reference in their entireties:

U.S. Pat. No. 7,999,161, titled “Laser-Activated Nanothermolysis Of Cells” filed Jul. 23, 2007;

U.S. Pat. No. 9,289,191, titled “System and method for Acquiring Optoacoustic Data and Producing Parametric Maps Thereof”, and filed Jun. 13, 2012;

U.S. Pat. No. 9,517,055, titled “System And Method For Acquiring Optoacoustic Data And Producing Parametric Maps Using Subband Acoustic Compensation” filed Nov. 25, 2013;

U.S. Pat. No. 9,724,072, titled “System And Method For Mixed Modality Acoustic Sampling” filed Dec. 13, 2013;

U.S. Pat. No. 9,456,805, titled “System And Method For Acquiring Optoacoustic Data And Producing Parametric Maps Using Interframe Persistent Artifact Removal” filed Dec. 19, 2013;

U.S. Publication 2016/0199037, titled “System And Method For Acquiring Optoacoustic Data And Producing Parametric Maps thereof” filed Mar. 22, 2016;

U.S. Publication 2017/0035388, titled “System And Method For Mixed Modality Acoustic Sampling” filed Oct. 18, 2016;

U.S. Pat. No. 9,792,686, titled “System And Method For Acquiring Optoacoustic Data And Producing Parametric Maps Using Subband Acoustic Compensation” filed Nov. 17, 2016;

U.S. Publication 2017/0296151, titled “System And Method For Mixed Modality Acoustic Sampling” filed Jun. 30, 2017;

U.S. Publication 2013/0109950, titled “Handheld Optoacoustic Probe” filed Nov. 2, 2011;

U.S. Publication 2016/0296121, titled “Handheld Optoacoustic Probe” filed May 2, 2016;

U.S. Pat. No. 8,686,335, titled “System And Method For Adjusting The Light Output Of An Optoacoustic Imaging System” filed Dec. 31, 2011;

U.S. Pat. No. 9,528,936, titled “System And Method For Adjusting The Light Output Of An Optoacoustic Imaging System” filed Mar. 31, 2014;

U.S. Publication 2017/0108429, titled “System And Method For Adjusting The Light Output Of An Optoacoustic Imaging System” filed Dec. 27, 2016;

U.S. Pat. No. 9,330,452, titled “Statistical Mapping In An Optoacoustic Imaging System” filed Mar. 11, 2013;

U.S. Pat. No. 9,836,838, titled “Statistical Mapping In An Optoacoustic Imaging System” filed May 3, 2016;

U.S. Publication 2018/0061050, titled “Statistical Mapping In An Optoacoustic Imaging System” filed Nov. 6, 2017;

U.S. Pat. No. 9,610,043, titled “System And Method For Producing Parametric Maps Of Optoacoustic Data” filed Jun. 13, 2012;

U.S. Publication 2017/0100040, titled “System And Method For Producing Parametric Maps Of Optoacoustic Data” filed Dec. 21, 2016;

U.S. Publication 2013/0338501, titled “System And Method For Storing Data Associated With The Operation Of A Dual Modality Optoacoustic/Ultrasound System” filed Jun. 13, 2012;

U.S. Publication 2013/0338475, titled “Optoacoustic Imaging System With Fiber Optic Cable” filed Jun. 13, 2012;

U.S. Publication 2014/0194723, titled “Multi-Layer Coating For Optoacoustic Probe” filed Jan. 13, 2014;

U.S. Publication 2017/0150890, titled “Optoacoustic Probe With Multi-Layer Coating” filed Jan. 31, 2017;

U.S. Pat. No. 9,615,750, titled “Methods And Compositions For Carrier Agents And Clearing Agents Used In Optoacoustic Imaging Systems” filed Jun. 14, 2012;

U.S. Publication 2013/0116538, titled “Optoacoustic Imaging Systems And Methods With Enhanced Safety” filed Oct. 19, 2012;

U.S. Publication 2015/0297090, titled “Optoacoustic Imaging Systems And Methods With Enhanced Safety” filed Jan. 23, 2015;

U.S. Publication 2013/0289381, titled “Dual Modality Imaging System For Coregistered Functional And Anatomical Mapping” filed Nov. 2, 2012;

U.S. Pat. No. 9,757,092, titled “Method For Dual Modality Optoacoustic Imaging” filed Nov. 2, 2012;

U.S. Publication 2014/0039293, titled “Optoacoustic Imaging System Having Handheld Probe Utilizing Optically Reflective Material” filed Jan. 22, 2013;

U.S. Publication 2017/0014101, titled “Dual Modality Imaging System For Coregistered Functional And Anatomical Mapping” filed Sep. 27, 2016;

U.S. Publication 2013/0303875, titled “System And Method For Dynamically Varying The Angle Of Light Transmission In An Optoacoustic Imaging System” filed Nov. 2, 2012;

U.S. Pat. No. 9,445,785, titled “System And Method For Normalizing Range In An Optoacoustic Imaging System” filed Dec. 21, 2012;

U.S. Pat. No. 9,282,899, titled “System And Method For Detecting Anomalous Channel In An Optoacoustic Imaging System” filed Dec. 21, 2012;

U.S. Publication 2014/0005544, titled “System And Method For Providing Selective Channel Sensitivity In An Optoacoustic Imaging System” filed Dec. 21, 2012;

U.S. Publication 2016/0317034, titled “System And Method For Providing Selective Channel Sensitivity In An Optoacoustic Imaging System” filed Jul. 11, 2016;

U.S. Pat. No. 9,445,786, titled “Interframe Energy Normalization In An Optoacoustic Imaging System” filed Jan. 22, 2013;

U.S. Publication 2017/0000354, titled “Interframe Energy Normalization In An Optoacoustic Imaging System” filed Sep. 19, 2016;

U.S. Publication 2014/0206978, titled “Probe With Optoacoustic Isolator” filed Jan. 22, 2013;

U.S. Pat. No. 9,743,839, titled “Playback Mode In An Optoacoustic Imaging System” filed Mar. 15, 2013;

U.S. Publication 2017/0332916, titled “Playback Mode In An Optoacoustic Imaging System” filed Jul. 27, 2017;

U.S. Pat. No. 9,398,893, titled “System And Method For Diagnostic Vector Classification Support” filed Mar. 11, 2014;

U.S. Pat. No. 10,026,170, titled “System And Method For Diagnostic Vector Classification Support” filed Jul. 19, 2016

U.S. Application number 16/022,138, titled “System And Method For Diagnostic Vector Classification Support” filed Jun. 28, 2018;

U.S. Pat. No. 9,730,587, titled “Diagnostic Simulator” filed Mar. 15, 2013;

U.S. Publication 2017/0332915, titled “Diagnostic Simulator” filed Jul. 27, 2017;

U.S. Pat. No. 8,823,928, titled “Light Output Calibration In An Optoacoustic System” filed Mar. 15, 2013;

U.S. Pat. No. 9,163,980, titled “Light Output Calibration In An Optoacoustic System” filed Jul. 11, 2014;

U.S. Pat. No. 9,814,394, titled “Noise Suppression In An Optoacoustic System” filed Mar. 15, 2013;

U.S. Publication 2018/0078144, titled “Noise Suppression In An Optoacoustic System” filed Nov. 13, 2017;

U.S. Pat. No. 9,733,119, titled “Optoacoustic Component Utilization Tracking” filed Mar. 15, 2013;

U.S. Publication 2017/0322071, titled “Optoacoustic Component Utilization Tracking” filed Jul. 27, 2017;

U.S. Publication 2015/0101411, titled “Systems And Methods For Component Separation In Medical Imaging” filed Oct. 13, 2014;

U.S. Publication 2015/0305628, titled “Probe Adapted To Control Blood Flow Through Vessels During Imaging And Method Of Use Of Same” filed Feb. 27, 2015

U.S. Publication 2016/0187481, titled “Opto-Acoustic Imaging System With Detection Of Relative Orientation Of Light Source And Acoustic Receiver Using Acoustic Waves” filed Oct. 30, 2015.

The terms “optoacoustic image” and “OA image” refer to an image captured by an imaging system that utilizes transmitted light at one or more frequencies into a tissue site and receives optoacoustic return signals at an optoacoustic transducer that are processed to generate optoacoustic image data that is converted into the OA image. In example embodiments, the optoacoustic return signals are in a range between 250 Hz and 2.5 MHz.

The term “ultrasound image” refers to an image captured by an imaging system that utilizes transmitted light at one or more frequencies into a tissue site and receives ultrasound return signals at an ultrasound transducer that are processed to generate ultrasound image data that is converted into the ultrasound image. In example embodiments, the ultrasound return signals are in a range between 200 MHz and 25 MHz.

As used herein the term “light” shall refer to any and all electromagnetic radiation, including but not limited to UV radiation, visible light, infrared radiation, etc. Light as used herein is in no way limited to the visible spectrum. Light may include characteristics including polarization, wavelength, frequency, etc. When a characteristic of light is changed, enhanced, diminished, altered, etc. the light may be considered converted, changed, enhanced, diminished, altered, etc.

The term “tissue site” broadly refers to locations or targets of animal and human tissues and organs such as, for example, breast tissue. A tissue site may contain a variety of different “tissue structures” that may include, for example, tumors, blood vessels, tissue layers, and components of blood. As described below, a sinogram may contain a sample recording of acoustic activity occurring over a period of time in response to one or more light events impinging on the tissue site. The acoustic activity captured in the sinogram may include an optoacoustic response, i.e., the acoustic signal that is created as a result of the electromagnetic energy being absorbed by materials within the tissue site such as, for example, various tissue structures that absorb the electromagnetic energy. These optical signals result from the release of thermo-elastic stress confinement within the tissue structures in response to the light events.

A dual imaging probe is provided that combines an ultrasound probe that captures higher frequency return signals (e.g. 20 Mega Hertz (MHz) and 25 MHz), and an optoacoustic probe that captures lower frequency return signals (e.g. 250 Hertz (Hz) and 2.5 MHz). As a result, both ultrasound imaging and optoacoustic imaging can be obtained during a single insertion into a body cavity at a tissue site without having to reinsert a different probe. To provide the dual imaging functionality, a first transducer for obtaining the higher frequency return signals, and a second transducer for obtaining the lower frequency return signals are both placed on a probe sidewall at a distal end of the probe. In one example, the first transducer is placed 180 degrees from the second transducer. Alternatively, the second transducer can then be staggered down from the first transducer to provide more space when aligned on the same sidewall instead of providing a 180 degree spacing. In either instance, a user can obtain imaging data with the high frequency transducer for ultrasound, and when ready to start OA imaging the user rotates the probe 180 degrees, or move the probe laterally, for OA imaging without the need to have two probes and replace each as needed.

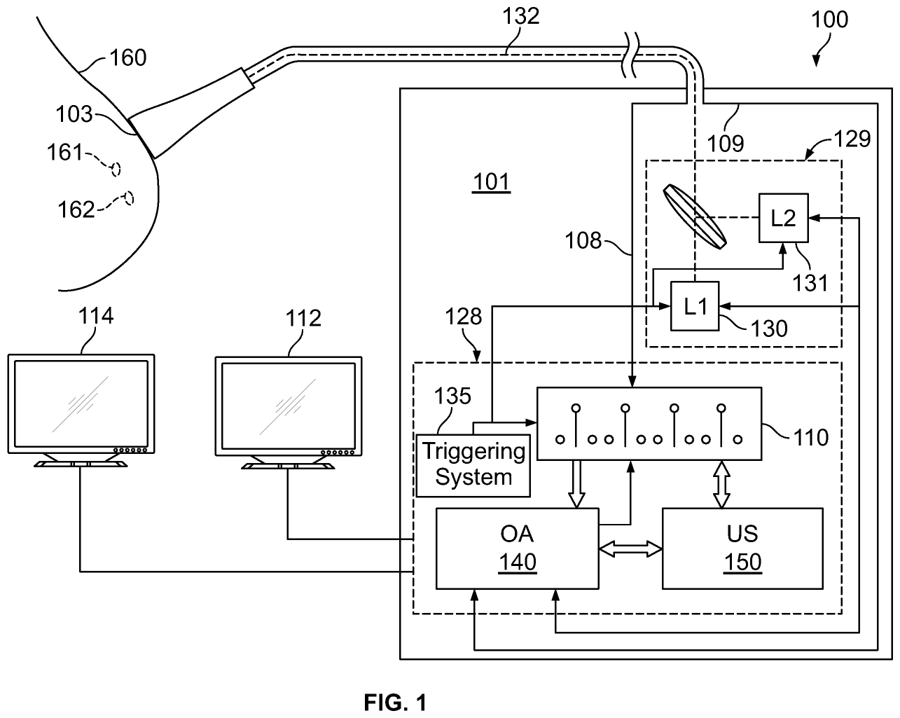

Turning to FIG. 1, generally, device 100 for dual imaging that may be employed as multimodality, combined optoacoustic and ultrasound system. In an embodiment, the device 100 includes a probe 102 for dual imaging that is connected via a light path 132 and an electrical path 108 to a system chassis 101. Within the system chassis 101 is housed a laser assembly 129 that utilizes one or more lasers to emit the light of the light path, and a computing subsystem 128.

The computing subsystem 128 includes one or more computing components for ultrasound control and analysis and optoacoustic control and analysis; these components may be separate, or integrated. In an embodiment, the computing subsystem 128 is or include a microcontroller. The computing subsystem in one example comprises a relay system 110, a triggering system 135, an optoacoustic processing and overlay system 140 and an ultrasound instrument 150. In one embodiment, the triggering system 135 is configured to actuate and control operation of a laser 130 to emit light.

In an embodiment, the laser assembly 129 is capable of producing pulses of light of at least two different wavelengths, and at varying frequencies. In one example the pulses of light can be provided to result in optoacoustic return signals having a lower frequency range, such as between 250 Hertz (Hz) and 2.5 Mega Hertz (MHz), and also result in ultrasound return signals having a higher frequency range such as between 20 MHz and 25 MHz. In this manner, the probe 102 can include dual functionality for providing both optoacoustic images and ultrasound images without the need of changing probes.

The output of the laser 130 of the laser assembly 129 is delivered to the probe 102 via the light path 132. The laser light is emitted on a tissue site 160, or targeted area of a volume, such as a breast or prostrate, resulting in soundwaves being formed as a result of the laser bouncing of objects. These soundwaves are then utilized to provide both optoacoustic images and ultrasound images of the targeted area of the volume for analysis.

One or more displays 112, 114, which may be touch screen displays, are provided for displaying images and all or portions of the device 100 user interface. The display images may include a first image that is an optoacoustic image, and a second image that is an ultrasound image. One or more other user input devices (not shown) such as a keyboard, mouse, and various other input devices (e.g., dials and switches) may be provided for receiving input from an operator.

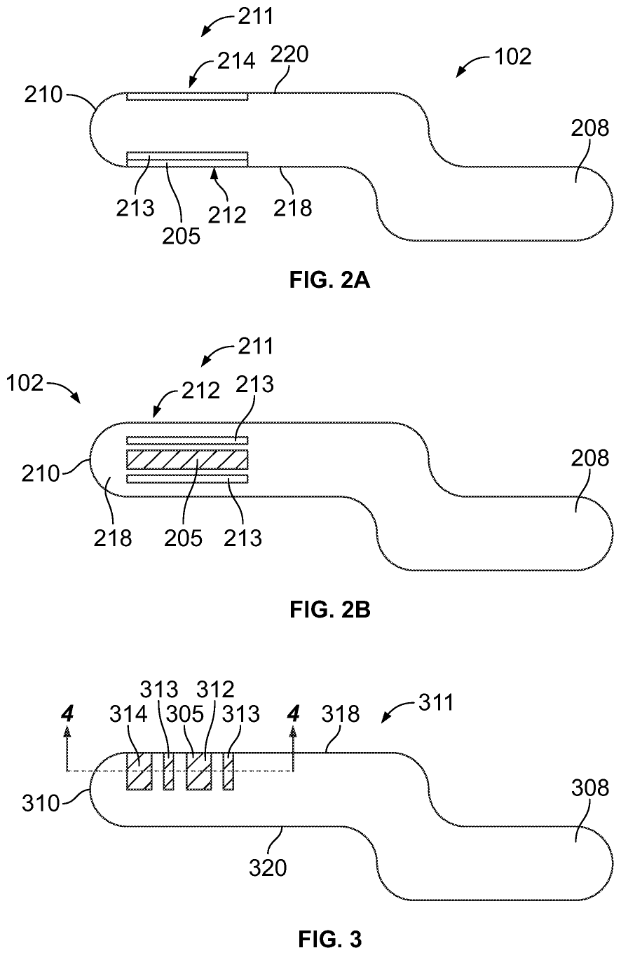

Turning now to FIG. 2A and 2B, The probe 102 extends from a distal end 208 to a proximal end 210. At the distal end 208 of the probe 102 along a side wall is a transducer assembly 211 that can include is a first transducer 212. The first transducer 212 in one example is covered by an acoustic lens 205. In another example, the first transducer 212 is configured to receive optoacoustic return signals having a lower frequency range, such as between 250 Hz and 2.5 MHz. In addition, in one example, at least one light bar 213 is positioned adjacent to the first transducer on the sidewall of the probe 102. The light bars 213 are provided to generate signals that are obtained by the first transducer 212 for imaging. In one example, a first light bar and second light bar are provided spaced on either side of the first transducer as illustrated in FIG. 2B.

Additionally, the transducer assembly 211 can include a second transducer 214 also located on or at a sidewall of the probe 102. In one example, the second transducer 214 is configured to receive ultrasound return signals having a higher frequency range such as between 20 MHz and 25 MHz. In particular, in an embodiment when the probe 102 is utilized as a prostrate probe, the size of a prostate probe is as small as possible for comfort. The limiting factor for how small a prostrate probe can be is based on the transducers at the distal end of the probe. In one example, the probe 102 has a larger protrusion at the distal end 208 of the probe to make space for both the first transducer 212 and second transducer 214, and then the body of the probe thins out for the length of the probe. This is because the housing only needs to hold the wires, or flex, of co-ax connecting the first transducer 212 and second transducer 214 to the system. Therefore, there is more space in the area directly adjacent to the first transducer 212 and second transducer 214 is extended up through the handle.

To this end, in one embodiment, (e.g. FIGS. 2A-2B), the first transducer 212 and second transducer 214 are spaced from one another on the probe sidewall. In particular, in the example embodiment of FIG. 2A, the probe sidewall includes a first arcuate side 218 and second arcuate side 220 opposite the first arcuate side 218. In one example, the first transducer 212 and light bars 213 are located on the first arcuate side 218, while the second transducer 214 is located on the second arcuate side 220. In one example, the first transducer 212 is located 180° from the second transducer 214. Optionally, when the first transducer is in a first position for receiving the optoacoustic return signals from the tissue site 160 the second transducer 214 is not in a spatial location to receive ultrasound return signals from the tissue site. However, when rotating the probe 102 180° to a second position, the second transducer 214 is then in a spatial location to receive ultrasound return signals from the tissue site 160, while the first transducer 212 is not in a spatial position to receive the optoacoustic return signals from the tissue site 160. Therefore, in the embodiment of FIGS. 2A-2B, the functionality of the probe 102 may be changed from a probe 102 that is utilized to provide a first image that is an optoacoustic image, to a probe 102 that is utilized to provide a second image that is an ultrasound image by rotating the probe 102 180°. As a result, the probe does not have to be removed to obtain both the first image and second image. Optionally, a second probe does not have to be provided, reducing time spent for examination. This allows the user to image with the high frequency second transducer 214 for ultrasound, and when ready to start optoacoustic imaging, the user can simply rotate the probe 102 180° degrees and begin imaging in optoacoustic without the need to have two probes and replace each as needed.

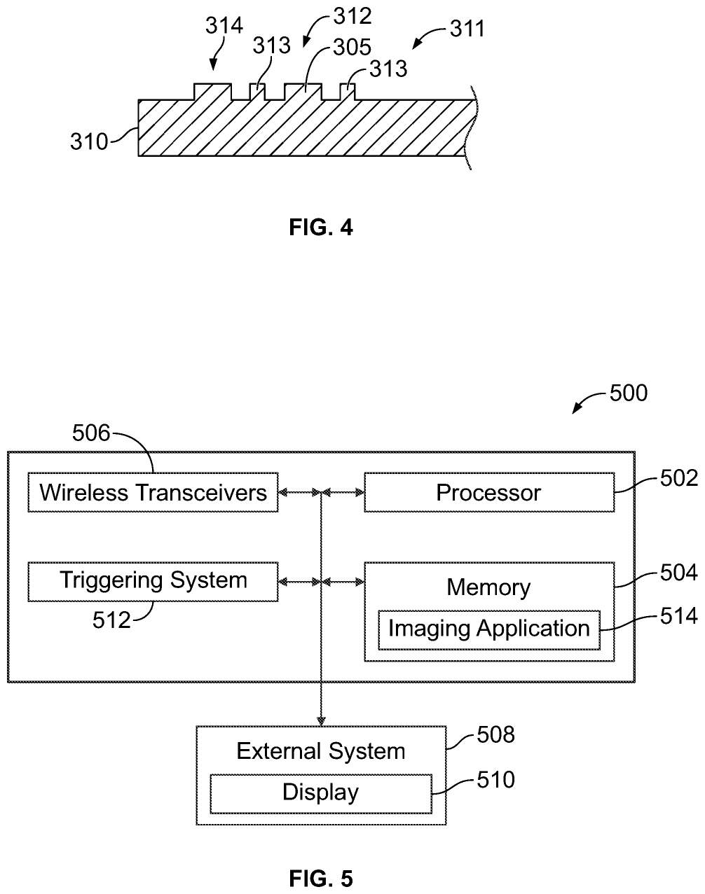

FIGS. 3-4 illustrate an alternative to the embodiment of FIGS. 2A-2B. In particular, in the embodiment of FIGS. 3-4 the transducer assembly 311 also extends from a distal end 308 to a proximal end 310. In this example, the first transducer 312 and second transducer 314 can be aligned on a single arcuate side 318, 320 such that the second transducer 314 can extend further distally than the first transducer 312. In example embodiments, similar to the embodiment of FIGS. 2A-2B the first transducer 312 can be is configured to receive optoacoustic return signals having a lower frequency range, such as between 250 Hz and 2.5 MHz, while the second transducer can be configured to receive ultrasound return signals having a higher frequency range such as between 20 MHz and 25 MHz. The first transducer 312 may also include an acoustic lens 305, and have light bars 313 positioned on either side in spaced relation. In one example, the second transducer 314 is staggered down from the first transducer 312 to where there is more space that would be occupied by only wires. The housing for ultrasound imaging only needs to hold wires, or flex, of co-axial connecting the second transducer 314 to the system. Therefore, there is more space in the area directly adjacent to the second transducer 314 through the handle of the probe 102.

With reference back to FIG. 1, the probe 102 also can include one or more optical windows 103 through which the light is carried on light path 132 can be transmitted to the surface of a tissue site 160, for example, a three-dimensional volume. Optionally, the probe 102 may be placed in close proximity with organic tissue, phantom, or other tissue site 160 that may have one or more inhomogeneities 161, 162, such as e.g., a tumor, within. An ultrasound gel (not shown) or other material may be used to improve acoustic coupling between the probe 102 and the surface of the tissue site 160 and/or to improve optical energy transfer.

FIG. 5 illustrates a schematic block diagram of a microcontroller 500. In one example, the microcontroller 500 is the computing subsystem 128 of FIG. 1. Alternatively, the microcontroller 500 is a component of the computing subsystem 128 of FIG. 1. The microcontroller 500 includes one or more processors 502, and a memory 504 coupled to the one or more processors 502. The memory 504 store instructions that can be executed by the one or more processors 502. The instructions may include instructions to perform processes and methods as described herein. The microcontroller 500 can also include a transceiver 506 for communicating with components and systems of the probe, along with external systems 508. The external systems 508 include imaging systems that have a display 510 in order to display images, including optoacoustic images, ultrasound images, or the like.

The microcontroller 500 also includes a triggering system 512 that is coupled to a light source for providing the light for the probe. In one example, the microcontroller can vary light characteristics including wavelength, intensity, frequency, etc. of the light emitted by the light source utilizing the triggering system 512. Alternatively, the microcontroller 500 or external device 508 may be utilized to vary the light characteristics.

In one example, stored within the memory 504 is an imaging application 514. The imaging application 514 includes instructions and is configured to convert optoacoustic return signals into optoacoustic image data that can be provided on a display as a first image, and is configured to convert ultrasound return signals into ultrasound image data that can be provided on the display as a second image. In one example, the imaging application 514 converts return signals received that are in the range of frequency range between 250 Hertz (Hz) and 2.5 Mega Hertz (MHz) to convert the optoacoustic return signals into a first image. In addition, the imaging application 514 converts the ultrasound return signals having a frequency range between 20 MHz and 25 MHz to convert the ultrasound return signals into a second image. In addition, the imaging application 514 is configured to actuate the triggering system, including to vary light characteristics during imaging. In another example, the imaging application is configured to store images, communicate images to remote devices, etc.

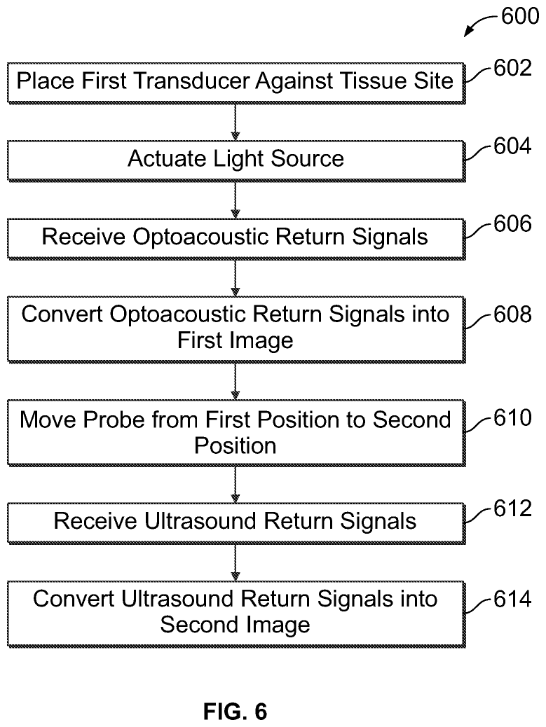

FIG. 6 illustrates a schematic flow block diagram of a method 600 of imaging a tissue site with a probe for dual imaging. In one example, the probe of FIGS. 1-4 is the dual imaging probe. In another example, the imaging application 514 of FIG. 5 includes the instructions for executing at least some of the steps of the method 600.

At 602, a first transducer on a distal end of the dual imaging probe is placed against a tissue site. In one example, the first transducer is spaced from the tissue site on probe sidewall while an optical window engages the tissue site. The tissue site can be a prostrate, breast, or the like.

At 604, one or more processors actuate a light source for emitting light on the tissue site. In one example, the one or more processors actuate the light source based on signals received from a trigger system controlled by a clinician. In another example, the light source includes a laser. In one example, the light source includes a first laser emitting light at a first wavelength, and a second laser emitting light at a second wavelength. The light in one embodiment travels along a light path and exits an optical window.

At 606, the first transducer is positioned and receives optoacoustic return signals during a first interval. In particular, the first interval occurs during a time a clinician is obtaining optoacoustic image data. The interval begins when the clinician begins positioning the probe to obtain optoacoustic image data, and ends when the clinician stops attempting to obtain optoacoustic image data. As a result, the first transducer is positioned to receive the light reflected off inhomogeneities within the tissue site accordingly. In one example, the first transducer is spaced from a second transducer, such that each of the first transducer and second transducer can be positioned to receive return signals from a first light. As such, the first transducer and second transducer must be positioned to receive the return signals during operation of the probe. Alternatively, the first transducer and second transducer can be stacked on one another so that the probe does not have to be moved and repositioned to obtain ultrasound image data after receiving optoacoustic image data.

At 608, the one or more processors convert the optoacoustic return signals into a first image. In one example, the first image is an optoacoustic image. In particular, the optoacoustic return signals received by the first transducer are analyzed and formed into image data that can be displayed on an output or screen of the probe, an external system, etc.

At 610, the dual imaging probe is moved from a first position to a second position to place a second transducer on the distal end against the tissue site. Again, similar to 602, the second transducer can be spaced from the tissue site on the probe sidewall while an optical window engages the tissue site. In one example, the second transducer extends further distally than the second transducer such that lateral movement results in dual imaging. In another example, the dual imaging probe is rotated 180° from the first position to the second position where in the first position the first transducer is positioned to receive optoacoustic signals, and in the second position, the second transducer is positioned to receive ultrasound signals. Rotating the dual imaging probe does not comprise withdrawing the dual imaging probe from the tissue site. In particular, the dual imaging probe allows the first transducer to receive optoacoustic signals and a second transducer to be rotated or moved into place to receive ultrasound signals during the same time period and while still inserted for engagement, or engaging the tissue site. As such, a second probe does not need to be utilized saving time and obtaining additional imaging data.

At 612, the second transducer receives ultrasound return signals during a second interval. In one example, the second interval is the time in which a physician attempts to obtain ultrasound data, including the repositioning of the probe through rotation to obtain the ultrasound data until the clinician stops utilizing the triggering system to obtain ultrasound image data.

At 614, the one or more processors convert the ultrasound return signals into a second image. In one example, the second image is an ultrasound image. In particular, the ultrasound return signals received by the first transducer are analyzed and formed into image data that can be displayed on an output or screen of the probe, an external device, etc.

As used in this description and in the following claims, “a” or “an” means “at least one” or “one or more” unless otherwise indicated. In addition, the singular forms “a”, “an”, and “the” include plural referents unless the content clearly dictates otherwise. Thus, for example, reference to a composition containing “a compound” includes a mixture of two or more compounds. As used in this specification and the appended claims, the term “or” is generally employed in its sense including “and/or” unless the content clearly dictates otherwise.

Unless otherwise indicated, all numbers expressing quantities of ingredients, measurement of properties and so forth used in the specification and claims are to be understood as being modified in all instances by the term “about,” unless the context clearly dictates otherwise. Accordingly, unless indicated to the contrary, the numerical parameters set forth in the foregoing specification and attached claims are approximations that can vary depending upon the desired properties sought to be obtained by those skilled in the art utilizing the teachings of the present invention. At the very least, and not as an attempt to limit the scope of the claims, each numerical parameter should at least be construed in light of the number of reported significant digits and by applying ordinary rounding techniques. Any numerical value, however, inherently contains certain errors necessarily resulting from the standard deviations found in their respective testing measurements.

Those skilled in the art will recognize that the methods and systems of the present disclosure may be implemented in many manners and as such are not to be limited by the foregoing example embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible. Functionality may also be, in whole or in part, distributed among multiple components, in manners now known or to become known. Thus, myriad software/hardware/firmware combinations are possible in achieving the functions, features, interfaces, and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

Various modifications and alterations to the invention will become apparent to those skilled in the art without departing from the scope and spirit of this invention. It should be understood that the invention is not intended to be unduly limited by the specific embodiments and examples set forth herein, and that such embodiments and examples are presented merely to illustrate the invention, with the scope of the invention intended to be limited only by the claims attached hereto. Thus, while the invention has been particularly shown and described with reference to a preferred embodiment thereof, it will be understood by those skilled in the art that various changes in form and details may be made therein without departing from the spirit and scope of the invention.

What is claimed is:

1. A probe for dual imaging of a tissue site, the probe having a distal end operable to contact the tissue site and a proximal end, the probe comprising:
a light source configured to generate light that is transmitted along a light path to generate optoacoustic return signals and ultrasound return signals when the light reacts with the tissue site;
a transducer assembly including a first transducer on the distal end, and a second transducer on the distal end;
the first transducer configured to receive the optoacoustic return signals and having an acoustic lens provided over the first transducer;
the second transducer configured to receive the ultrasound return signals;
an optical window configured to carry light along the light path to the tissue site; and
a microcontroller including one or more processors, and a memory coupled to the one or more processors, wherein the memory stores program instructions, wherein the program instructions are executable by the one or more processors to:
convert the optoacoustic return signals from the first transducer into a first image; and
convert the ultrasound return signals from the second transducer into a second image.

2. The probe of claim 1, wherein the first transducer is spaced from the second transducer.

3. The probe of claim 2, wherein the first transducer is 180° from the second transducer.

4. The probe of claim 1, wherein the first transducer is stacked on the second transducer.

5. The probe of claim 1, wherein the optoacoustic return signals received by the first transducer have a frequency range between 250 Hertz (Hz) and 2.5 Mega Hertz (MHz), and the ultrasound return signals have a frequency range between 20 MHz and 25 MHz.

6. The probe of claim 1, wherein the light source is a laser.

7. The probe of claim 1, wherein the first transducer extends further distally than the second transducer.

8. The probe of claim 1, further comprising a triggering assembly coupled to the light source for actuating the light source.

9. A method of imaging a tissue site with a dual imaging probe comprising:
placing a first transducer on a distal end of the dual imaging probe against a tissue site;
actuating a light source for emitting light on the tissue site;
receiving, with the first transducer, optoacoustic return signals;
converting the optoacoustic return signals into an optoacoustic image;
rotating the dual imaging probe to place a second transducer on the distal end against the tissue site;
receiving, with the second transducer, ultrasound return signals; and
converting the ultrasound return signals into an ultrasound image.

10. The method of claim 9, wherein rotating the dual imaging probe comprises rotating the dual imaging probe 180°.

11. The method of claim 9, wherein the optoacoustic return signals received by the first transducer have a frequency range between 250 Hertz (Hz) and 2.5 Mega Hertz (MHz), and the ultrasound return signals have a frequency range between 20 MHz and 25 MHz.

12. The method of claim 11, wherein rotating the dual imaging probe does not comprise withdrawing the dual imaging probe from the tissue site.

13. A probe for dual imaging, the probe having a distal end operable to contact a tissue site and a proximal end, the probe comprising:
a light source configured to generate light that is transmitted along a light path to generate optoacoustic return signals and ultrasound return signals when the light reacts with the tissue site;
a transducer assembly including a first transducer on the distal end, and a second transducer on the distal end;
the first transducer configured to receive the optoacoustic return signals in a first position;
the second transducer configured to receive the ultrasound return signals in a second position;
an optical window configured to carry light along the light path to the tissue site; and
a microcontroller including one or more processors, and a memory coupled to the one or more processors, wherein the memory stores program instructions, wherein the program instructions are executable by the one or more processors to:
convert the optoacoustic return signals from the first transducer into a first image;
and convert the ultrasound return signals from the second transducer into a second image.

14. The probe of claim 13, wherein the first transducer is spaced from the second transducer.

15. The probe of claim 13, wherein the first transducer is 180° from the second transducer.

16. The probe of claim 15, wherein the probe rotates 180° between the first position and the second position.

17. The probe of claim 13, wherein the optoacoustic return signals received by the first transducer have a frequency range between 250 Hertz (Hz) and 2.5 Mega Hertz (MHz), and the ultrasound return signals have a frequency range between 20 MHz and 25 MHz.

18. The probe of claim 13, wherein the light source is a laser.

19. The probe of claim 13, wherein the first transducer extends further distally than the second transducer.

20. The probe of claim 13, further comprising a triggering assembly coupled to the light source for actuating the light source.
